(19) United States
(12) Patent Application Publication
(10) Pub. No.: US 2007/0293836 A1
(43) Pub. Date: Dec. 20, 2007

(54) High surface energy tampon

(75) Inventors: Margaret Henderson Hasse, Wyoming, OH (US); Linda Marie Bumpass, Crestview Hills, KY (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/454,507

(22) Filed: Jun. 16, 2006

(51) Int. Cl. A61F 13/20

(52) U.S. Cl. 604/385.18; 604/904; 604/363

(57) ABSTRACT

An improved absorbent tampon is disclosed which comprises a high surface energy outer surface that is more desirable to menses than the surface of vaginal tissue during menstruation. Thus, the surface energy of the outer surface of the tampon is greater than the surface energy of the vaginal cavity surface during menstruation. This makes the outer surface of the tampon of the present invention more desirable in a sense to the menses than the vaginal cavity surface, therefore reducing the occurrence of bypass leakage. This is accomplished without relying on body pressure and capillary absorption both of which have limited effectiveness and can cause negative comfort issues with the wearer.

### FIELD OF INVENTION

This invention relates to an improved absorbent tampon, which provides improved bypass leakage protection by having a tampon outer surface with high surface energy properties.

### BACKGROUND OF THE INVENTION

As the surface of the vaginal cavity is both hydrophilic and highly textured it provides an ideal surface for transporting bodily discharges for two reasons. First, the surface of the vaginal cavity is hydrophilic and has a high affinity for blood or other bodily discharges. Secondly, the blood is able to move freely on the textured surface within the naturally occurring channels that are present within the vaginal cavity. This provides quite a challenge for tampon products, because products that are worn predominantly within the vaginal cavity need to make close contact with the surface if they are to absorb effectively bodily discharges at or near their source.

The prior art has recognized various mechanisms by which tampons might fail to deliver superior performance. One such mechanism is often referred to in the art as “bypass” failure or “bypass” leakage. Bypass leakage occurs when the menses travels along the length and out of the vagina without contacting the tampon, i.e. the tampon fails to intercept the flowing menses. Another mechanism by which tampons permit bypass leakage is the failure of the tampon to acquire fluid as rapidly as fluid flows.

Current tampons rely on body pressure to prevent bypass leakage and capillary absorption to acquire menses as it contacts the outside surface of the tampon. Reliance on capillary absorption to acquire menses is problematic as once an area becomes wet, the capillary strength of that area increases and so fluid does not quickly wick to dry regions of the tampon. If more fluid approaches the tampon at the same spot, capacity may be already exhausted, thus decreasing the speed at which the tampon can acquire additional fluid. Further, high capillary absorption can sacrifice comfort by causing adhesion of the tampon outer surface to vaginal tissue.

Therefore, what is needed is a tampon that can reduce bypass leakage while maintaining comfort through a mechanism other than sole reliance on capillary absorption.

### SUMMARY OF THE INVENTION

A tampon having an outer surface is provided which comprises a compressed absorbent member, an overwrap substantially covering the compressed absorbent member and forming a tampon outer surface, and wherein the overwrap comprises a finish that includes at least one rewetter chemical agent such that the tampon outer surface has a surface energy greater than about 42 mN/m.

A tampon comprising a compressed absorbent member wherein the tampon comprises a finish that includes at least one rewetter chemical agent such that the tampon outer surface has a surface energy greater than about 42 mN/m.

### BRIEF DESCRIPTION OF THE DRAWINGS

While the specification concludes with claims particularly pointing out and distinctly claiming the subject matter that is regarded as forming the present invention, it is believed that the invention will be better understood from the following description taken in conjunction with the accompanying drawings, in which:

FIG. 1 is a perspective view of a tampon of the present invention incorporating a compressed absorbent member and an overwrap extending past the withdrawal end of the compressed absorbent member to form a skirt.

FIG. 2 is a perspective view of a tampon of the present invention incorporating a compressed absorbent member.

FIG. 3 is a plan view of an uncompressed tampon including absorbent material and an overwrap, which extends past the withdrawal end of the absorbent material.

FIG. 4 is an illustration of a spread liquid drop at 0.1 seconds after the liquid drop contacts the surface of a solid.

### DETAILED DESCRIPTION OF THE INVENTION

It has been discovered that during menstruation the pH of the human vaginal cavity surface increases. The increase in pH is associated with an increase in the surface energy of the vaginal cavity surface. The increase makes the vaginal cavity surface more desirable to menses, so that the menses adheres to and moves along the vaginal cavity surface for eventual removal from the body.

A tampon is in competition with the vaginal cavity surface for menses, and therefore to be more desirable than the vaginal cavity surface for such menses the outer surface of the tampon should have a higher surface energy. Tampons have relied on expansion to cover as much surface area of the vaginal cavity as possible in order to intercept fluid, but with a lower surface energy an inserted tampon can absorb only through capillary absorption the fluid that happens to flow to its surface. Because tampons are limited in size by comfort considerations, even the largest tampons do not expand to cover the entire vaginal surface, thus allowing the opportunity for leakage from bypass flow.

The tampon of the present invention comprises an outer surface with high surface energy relative to the vaginal cavity surface energy such that menses on the surface of vaginal tissue tends to wet out the outer surface of the tampon during menstruation. The surface energy difference makes the outer surface of the tampon of the present invention more desirable in a sense to the menses than the vaginal cavity surface, thereby causing menses to go into the tampon, reducing the occurrence of bypass leakage. This is accomplished without relying entirely on the capillary absorption of tampon fibers, which have limited effectiveness and, as stated previously, can cause negative comfort issues with the wearer.

While not being limited to theory it is believed that the improved surface energy characteristics of the outer surface of the tampon of the present invention are not determined by the fiber characteristics such as basis weight or diameter, but rather by the addition of at least one finish which includes a rewetter chemical agent to the outer surface of the tampon in amounts to provide high surface energy. An example of an overwrap with high surface energy is an overwrap made from a nonwoven web having a finish, for example, product no. 053YLCD09U manufactured by BBA Corporation of Washougal, Washington.

Definitions

Within the scope of this specification, each term or phrase below will include the following definition or definitions. These terms, however, may be defined further with additional language in other portions of the specification and/or the depiction in the figures.

As used herein the term “tampon” or “catamenial tampon” refers to any type of absorbent structure that is inserted into the vaginal canal for the absorption of fluid there from. Tampons have a length, a width, a longitudinal axis and a transverse axis. The tampon's length can be measured from the insertion end to the withdrawal end along the longitudinal axis. A typical compressed tampon is 30-60 mm in length. A tampon may be straight or non-linear in shape, such as curved along the longitudinal axis. The width of a tampon, unless otherwise stated in the specification, corresponds to the largest cylindrical cross-section along the length. A typical compressed tampon is 8-20 mm wide. While the tampon may be compressed into a substantially cylindrical configuration, other shapes are possible. These shapes may include shapes having a cross section that may be described as rectangular, triangular, trapezoidal, semi-circular, hourglass, or other suitable shapes.

As used herein “absorbent material” refers to the uncompressed absorbent member of a tampon without the overwrap material prior to compression to form the compressed absorbent member. The absorbent material may be any suitable size and thickness suitable for compression into a tampon having a vaginally insertable shape. The absorbent material is generally square or rectangular, but other shapes such as trapezoidal, triangular, hemispherical, chevron and hourglass shaped are also acceptable. In one embodiment, size for absorbent material prior to compression may be from about 40 mm to about 100 mm in length and from about 40 mm to about 80 mm in width. In certain embodiments the range for the overall basis weight is from about 150 μm (g/m²) to about 1,000 gsm.

The absorbent material that comprises the compressed absorbent member may be constructed from fibrous materials. Such fibrous materials may include but are not limited to synthetic fibers, natural fibers or combinations thereof. The natural fibers may include but are not limited to cotton, wood pulp, flax, hemp and rayon such as GALAXY Rayon (a tri-lobed rayon structure) available as 6140 Rayon; or SARILLE L rayon (a round fiber rayon), both available from Kelheim Fibers of Kelheim, Germany, cotton, wood pulp, flax, and hemp. The synthetic fibers can include but are not limited to fibers such as polyester, polyolefin, nylon, polypropylene, polyethylene, polyacrylic, vinyl polyacetate, polyacrylate, cellulose acetate or bicomponent fibers such as bicomponent polyethylene and polypropylene fibers. Additional absorbent material include materials, such as peat moss, absorbent foams (such as those disclosed in U.S. Pat. No. 3,994,298 issued to DesMarais on Nov. 30, 1976, U.S. Pat. No. 5,795,921 issued to Dyer, et. al both incorporated by reference herein,) capillary channel fibers (such as those disclosed in U.S. Pat. No. 5,356,405 issued to Thompson, et. al incorporated by reference herein), high capacity fibers (such as those disclosed in U.S. Pat. No. 4,044,766 issued Kaczmarzk et al. Aug. 30, 1977 incorporated by reference herein), superabsorbent polymers or absorbent gelling materials (such as those disclosed in U.S. Pat. No. 5,830,543 issued to Miyake, et al incorporated by reference herein) may be incorporated into the tampon.

As used herein “hydrophilic” and “hydrophobic” have meanings well established in the art with respect to the contact angle of a drop of water on the surface of a material. For example, a material having a contact angle of greater than about 75 degrees may be considered hydrophobic, and a material having a contact angle of less than about 75 degrees may be considered hydrophilic.

The term “joined” or “attached” as used herein, encompasses configurations in which a first element is directly secured to a second element by affixing the first element directly to the second element; configurations in which the first element is indirectly secured to the second element by affixing the first element to intermediate member(s) which in turn are affixed to the second element; and configurations in which the first element is integral with the second element; i.e., the first element is essentially part of the second element.

Unless specifically stated otherwise, as used herein a first material is “substantially covering” or “substantially covers” a second material when the first material covers the second material and/or when the first material covers a third material, which covers the second material. In other words, a first material may “substantially cover” a second material regardless of whether a third material or even a fourth material is interposed between the first material and the second material. Therefore, as used herein, a first material is “substantially covering” or “substantially covers” a second material when the first material covers from at least about 50% to about 100% of the surface area of the second material regardless of whether a third material is interposed between the first material and the second material.

Certain embodiments of the tampon of the present invention may comprise a withdrawal means such as a string, cord or any other means as known in the art. The withdrawal means may be joined to any suitable location on the tampon and graspable for digital removal after use. In addition to a string or cord configuration, the withdrawal means may be other forms such as a ribbon, loop, tab, or the like. Withdrawal cords useful in the present invention may be made of any suitable material known in the prior art and include cotton and polyester. Additionally, the tampons of the present invention may also benefit from a secondary absorbent member. U.S. Pat. No. 6,258,075 to Taylor et al. entitled “Tampon with Enhanced Leakage Protection” describes tampons having a variety of secondary absorbent members in detail and U.S. Pat. No. 6,840,927 to Hasse et al. entitled “Tampon With Fluid Wicking Overwrap With Skirt Portion.”

The tampon of the present invention may be inserted digitally or with the use of an applicator. Any of the currently available tampon applicators may also be used for insertion of the tampon of the present invention. Such applicators are typically of a “tube and plunger” type arrangement and may be plastic, paper, or other suitable material. Additionally, a “compact” type applicator may be used.

As used herein “overwrap” refers to materials that substantially cover the exterior surface of the compressed absorbent member and/or the first surface and the second surface of the absorbent material and that contact and transport menses over the surface of the tampon prior to absorption of the menses into the compressed absorbent member and/or the first surface and the second surface of the absorbent material. In certain embodiments the overwrap may substantially cover the exterior surface of the compressed absorbent member and/or the first and second surface of the absorbent material as well as, any interior surfaces or interior regions due to the folding or rolling of the absorbent material during compression.

The overwrap may be formed from nonwoven fibrous materials or apertured films. The nonwoven fibrous materials may comprise fibrous materials such as natural fibers, synthetic fibers or blends of natural and synthetic fibers. Natural fibers include but are not limited to rayon, cotton, wood pulp, flax, and hemp. Synthetic fibers can include but are not limited to fibers such as polyester, polyolefin, nylon, polypropylene, polyethylene, polyacrylic, vinyl polyacetate, polyacrylate, cellulose acetate or bicomponent fibers such as bicomponent polyethylene and polypropylene fibers. In one embodiment the overwrap comprises bicomponent polyethylene and polypropylene fibers wherein the fibers have a basis weigh of about 18 gsm (grams per square meter). The blend of fibers forming the overwrap can be made by any number of techniques such as being spunbond or carded. Commonly, carded webs that are hydroentangled, thermally bonded, and resin bonded all have application.

In embodiments that begin with providing a compressed absorbent member, the overwrap may be wrapped around a longitudinal axis or a transverse axis of the compressed absorbent member. The overwrap is positioned on the compressed absorbent member such that the overwrap substantially covers the exterior surface of the compressed absorbent. In embodiments that begin with providing a compressed absorbent member, the overwrap may be joined or applied to the compressed absorbent member subsequent to compression. Overwraps applied subsequent to compression should be extensible such that the tampon will be able to expand within the vagina.

The overwrap may be joined to itself, another overwrap, the compressed absorbent member or to the absorbent material. Such bonding may extend continuously along the length of attachment or it may be applied in a “dotted” fashion at discrete intervals. Methods of bonding include thermally bonding, fusion bonding, or any other suitable means known in the art for joining such materials. Alternatively, the overwrap may be joined to the absorbent material by stitching. Such stitching may use natural or synthetic thread.

The compressed absorbent members, absorbent materials and overwraps in addition to comprising a finish, which includes a rewetter chemical agent to increase the surface energy of the outer surface of the tampon may have one or more other finishes applied to their fibers as a means of enhancing their performance or aesthetic properties. Performance properties may include functional characteristics such as moisture regain and transport, absorbency or repellency and frictional behavior. Aesthetic properties may include various characteristics such as appearance, surface texture, color, and odor. The finishes applied may include one or more chemical agents, which provide the performance or aesthetic properties enhancing means in the finishes. Chemical agents include but are not limited to chemical agents such as rewetters, antistats, softeners, lubricants, optical brighteners or mixtures thereof. In certain embodiments the finishes may be applied as coatings to the fiber surfaces of fibers in the overwraps, absorbent materials and/or compressed absorbent members or may be added to the fibers during their formation or by any other manner known to one of ordinary skill in the art.

Rewetters are chemical agents that may be used to impart hydrophilicity to the compressed absorbent members, absorbent materials and overwraps. Rewetters increase the surface energy of the fibers of the compressed absorbent members, absorbent materials and overwraps. For hydrophobic fibers the treatment can render them more hydrophilic, and facilitates the movement and penetration of the menses into the outer surface of the tampon. Examples of rewetters include but are not limited to surfactants such as nonionic, anionic surfactants, amphoteric surfactants, ampholytic surfactants, cationic surfactants or mixtures thereof.

Antistats improve the conductivity of the fibers, coat the fiber with a thin layer of material that will attract a thin layer of moisture, and finish the fabric such that it holds a charge opposite to that normally accumulated on the fiber to neutralize the static charge. Examples of antistats include but are not limited to conductive carbon, metallic particles, polyamines, polyethoxylated amine, ammonium salts, carboxylic salts, quaternary ammonium salts, imidazoles, fatty amides, phosphates, phosphate esters, sulfonates, sulfates, phosphonates, glycols, ethoxylated fatty acids, ethoxylated fatty alcohols, sorbitan fatty acid esters or mixtures thereof.

Softeners may be applied to the fibers to improve their aesthetic and functional characteristics. The drape, abrasion resistance, sewability and tear strength of the compressed absorbent members, absorbent materials and overwraps can be improved with the addition of a softener. The softeners reduce the coefficient of friction between the fibers. There are different types of softeners that include but are not limited to anionic softeners such as sulfates or sulfonates, cationic softeners such as amines and quaternary amines and nonionic softeners such as silicones, ethylene oxide derivatives, hydrocarbon waxes or mixtures thereof.

Lubricants may be applied as processing aids to help in stretching or to improve the processability of the compressed absorbent members and overwraps. Lubricants impart the same properties as softeners such as the reduction in the coefficient of friction, but specifically reduce fiber friction. Examples of lubricants include but are not limited to sulphonated oils, oil emulsions, silicones, esters, polyethylene dispersions, fatty acid soaps or mixtures thereof.

Optical brighteners or fluorescent whitening agents are chemical agents that may be used like dyes or pigments to add brightness to the compressed absorbent members, absorbent materials and overwraps. They are colorless but can absorb UV light and reemit it to the visible range usually as a blue or blue-green, and can produce very white fabrics or brighten colored fabrics. An example of an optical brightener is titanium oxide (TiO₂).

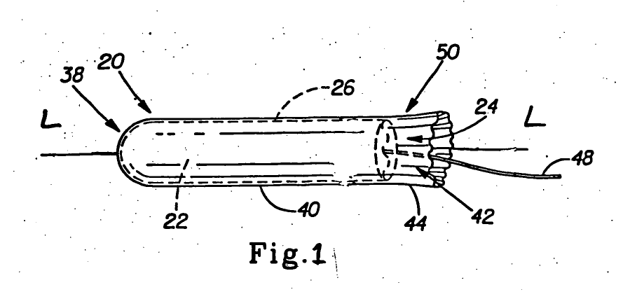

An embodiment of the tampon 20 of the present invention with an overwrap 40 is shown in FIG. 1. The compressed absorbent member 22 has an exterior surface 26 and has an inner region 24. The compressed absorbent member 22 has an insertion end 38 opposed to a withdrawal end 42. In FIG. 1, the overwrap 40 substantially covers at least a portion of the exterior surface 26 of the compressed absorbent member 22. The tampon 20 has a longitudinal axis indicated by the line marked “L. In the embodiment shown in FIG. 1, a portion of the overwrap 44 extends beyond the withdrawal end 42 of the compressed absorbent member 22 to define a skirt portion 50. In this embodiment, the tampon 20 of the present invention includes a withdrawal cord 48. The outer surface of the tampon, i.e. overwrap can have a relatively high surface energy with respect to the surface energy of the vaginal cavity surface during menstruation as discussed previously.

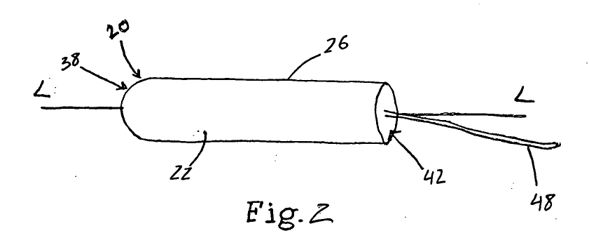

An embodiment of the tampon 20 of the present invention without an overwrap is shown in FIG. 2. The compressed absorbent member 22 has an exterior surface 26. The compressed absorbent member 22 has an insertion end 38 opposed to a withdrawal end 42. In this embodiment, the tampon 20 of the present invention includes a withdrawal cord 48. The tampon 20 has a longitudinal axis indicated by the line marked “L. The outer surface of the tampon, i.e. compressed absorbent member can have a relatively high surface energy with respect to the surface energy of the vaginal cavity surface during menstruation as discussed previously.

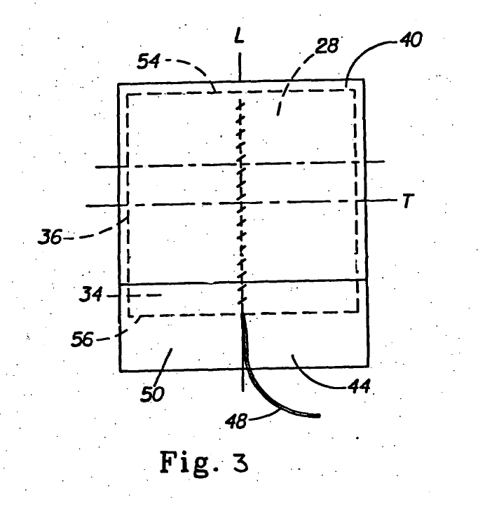

FIG. 3 depicts a plan view of an uncompressed tampon including the absorbent material 28 and overwrap 40. The absorbent material 28 has a first surface 34 opposed to the second surface 36 and an insertion end 54 opposed to a withdrawal end 56. The absorbent material 28 has both a longitudinal axis and a transverse axis indicated by the lines marked “L” and “T” respectively. The overwrap 40 substantially covers at least a portion of the first surface 34 and opposed second surface 36 of the absorbent material 28. In the embodiment shown in FIG. 3, a portion of the overwrap 44 extends beyond the withdrawal end 56 of the absorbent material 28 to define a skirt portion 50. In one embodiment, the absorbent material 28 includes a withdrawal cord 48. A tampon may be utilized in an uncompressed form. However, to form a compressed tampon, the absorbent material 28 and the overwrap 40 are typically compressed and optionally heat conditioned in any suitable conventional manner known in the art.

Tests were done to determine and compare the surface energy of the human vaginal cavity surface (during menstruation and non-menstruation), the outer surface of a tampon of the present invention (Sample A) and the outer surface of tampons that do not comprise a finish that includes at least one rewetter chemical agent (Samples B, C, and D). Surface energy of a substrate can be calculated, e.g. via the Fowkes equation from measured contact angles of drops of a fluid having a known surface energy. As contact angles of liquids are difficult to measure using human vaginal cavity surfaces in vivo, the determination of human vaginal cavity surface energy was done by using the vaginal cavity surfaces of fresh porcine vaginas, in certain embodiments within 1 to 2 days after removal. Porcine vaginas were selected as models for the determination of human vaginal cavity surface energy as they have a vaginal cavity surface that closely resembles that of a human female in structure, function and pH. The porcine vaginal cavity surface and the human vaginal cavity surface are similar in that they both consist of a stratified squamous epithelium and share similarities in lipid composition, histological condition and ultrastructural organization. The contact angles of Ethylene Glycol and Diiodomethane on the porcine vaginal cavity surface and tampon outer surfaces were measured at standard temperature and pressure (STP), and used to determine the surface energy of the porcine vaginal cavity surface and the outer surface of tampon samples A, B, C and D. Ethylene Glycol with a surface energy of 47.7 mN/m and Diiodomethane with a surface energy of 50.8 mN/m were used as both of their surface energies are known, and are in the range of the surface energy of human menses.

The samples used in Table 1 are as follows: Sample A is a tampon of the present invention with a 18 μm (grams per square meter) spunbond bicomponent polyethylene/polypropylene nonwoven overwrap available as 053YLCD09U, from BBA Group LLC, Washougal, Washington; Sample B is a TAMPAX tampon with a 18 μm, hydroentangled polyester nonwoven overwrap available as PGI 63010, from PGI (Polymer Group Inc.), North Charleston, S.C.; Sample C is a TAMPAX PEARL tampon with a 17 μm, carded and thermally bonded polyethylene/polypropylene nonwoven overwrap available as Sawabond 4313, from Sandler A G, Schwarzenbach, Germany; and Sample D is a TAMPAX tampon with a 35 μm hydroentangled rayon/polyester nonwoven overwrap available as PGI 92960, from PGI, North Charleston, S.C.

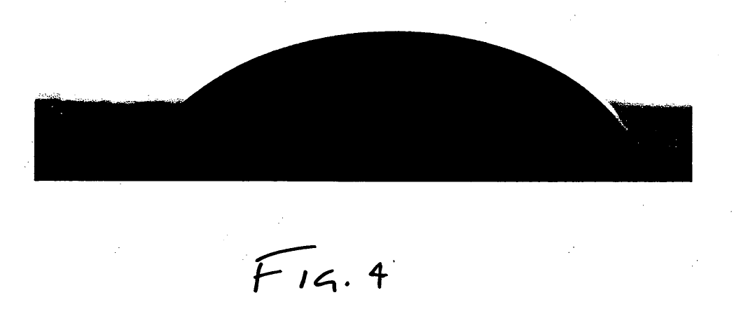

Using a pH probe, the pH of the porcine vaginal cavity surfaces was measured to be about 4.6, which is similar to the vaginal cavity surfaces of a non-menstruating human female. To simulate the increase in pH on the vaginal cavity surface that occurs in human females during menstruation, some of the porcine vaginal cavity surfaces were washed with about 4 liters of 0.1% KOH (potassium hydroxide) solution, followed by pure water, and afterward, measured as pH=6.9 using the same pH probe, which is similar to that of a menstruating human female. Pieces of the porcine vaginal cavity were then removed without disturbing the surface of the porcine vaginal cavity. The pieces were then laid flat on a surface and the contact angle of Ethylene Glycol and Diiodomethane on the porcine vaginal cavity surface measured optically. Initial spreading angles were measured and not angles which were influenced (diminished) by the liquids soaking into the samples. To accomplish this, high speed image analysis was used to take images of drops of Ethylene Glycol and Diiodomethane placed on the surface of the porcine vaginal cavities at a rate of 50 times per second. As shown in FIG. 4 small drops, of about 1.0 microliter were measured in the time frame of 0.1 seconds after droplet placement.

Optical measuring as described above was also used to measure the contact angles for the Ethylene Glycol and Diiodomethane on the outer surface of the tampon samples.

Test Surface as used in Table 1 refers to the porcine vaginal cavity surface or the outer surface of the tampon tested.

TABLE 1

| | Surface |
|---|---|
| | Energy |
| Test Surface | (mN/m) |
| PVCS | 32.6 |
| at pH = 4.6 | |
| PVCS | 42.3 |
| at pH = 6.9 | |
| Sample A | 45.2 |
| Sample B | 29.7 |
| Sample C | 31.4 |
| Sample D | 40.4 |

mN/m—milliNewtons per meter
PVCS—Porcine Vaginal Cavity Surface

The data in Table 1 shows a large difference in surface energy between the porcine vaginal cavity surface at pH=4.6 versus at pH=6.9, which are the pH's of the vaginal cavity surface of a non-menstruating and menstruating human female respectively. The surface energy of the porcine vaginal cavity surface at pH 6.9 rises nearly 10 mN/m, and the tampon of the present invention (Sample A) is the tampon that has an outer surface with a higher surface energy measurement (45.2 mN/m) than the vaginal cavity surface at pH=6.9 (42.3 mN/m). This demonstrates that only the tampon of the present invention (Sample A) has an outer surface with high surface energy relative to the vaginal cavity surface energy such that menses on the surface of vaginal cavity tends to wet out the outer surface of the tampon during menstruation.

To determine if a tampon of the present invention performed better than prior art tampons in bypass leakage protection a test was conducted. A test group comprised of seventy seven tampon users was provided with tampons corresponding to Sample A and Sample C and instructed to use them during their menstrual cycle. They were further instructed to use the tampons in a balanced pattern of alternate use (two Sample A tampons followed by two Sample C tampons and so on). Panelists recorded leakage incidents of any tampon Sample on a diary form. Used tampons were returned for weighing, and leakage incidents recorded. Only tampons at menses loads below the ultimate capacity of the tampon were used, and they represent tampons that range from dry to fully wetted. In the case of the tampons tested, tampons loaded with 0-6 grams of menses were compared. Beyond 6 grams normally the entire tampon has become wet, and surface energy no longer plays a role in performance because the surface energy of the tampon becomes the same as the surface energy of the blood being absorbed in to it. The results are set forth in Table 2 below.

TABLE 2

| Tampon | Leakage % |
|---|---|
| Sample A | 9 |
| Sample C | 16 |

The results show that the tampon of the present invention reduced the occurrence of leakage of menses by 44%, as compared to a conventional tampon, therefore, showing that a tampon with an outer surface having increased surface energy provides better protection against bypass leakage by being more desirable to menses than conventional tampons.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

It will be understood that the embodiment(s) described herein is/are merely exemplary, and that one skilled in the art may make variations and modifications without departing from the spirit and scope of the invention. All such variations and modifications are intended to be included within the scope of the invention as described hereinabove. Further, all embodiments disclosed are not necessarily in the alternative, as various embodiments of the invention may be combined to provide the desired result.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A tampon comprising:
compressed absorbent member;
an overwrap substantially covering the compressed absorbent member and forming a tampon outer surface; and
wherein the overwrap comprises a finish that includes at least one rewetter chemical agent such that the tampon outer surface has a surface energy greater than about 42 mN/m.

2. The tampon of claim 1 wherein the overwrap is a fibrous nonwoven material.

3. The tampon of claim 2 wherein the finish is disposed on fibers of the fibrous nonwoven.

4. The tampon of claim 1 wherein the finish comprises at least one chemical agent that is at least one of antistats, softeners, lubricants, optical brighteners or mixtures thereof

5. The tampon of claim 1 wherein the rewetter chemical agent comprises a surfactant that is at least one of a nonionic surfactant, anioinic surfactant, cationic surfactant, amphoteric surfactant, ampholytic surfactant or mixtures thereof.

6. The tampon of claim 1 wherein the overwrap extends beyond the withdrawal end of the compressed absorbent member.

7. The tampon of claim 1 wherein the overwrap comprises at least one of nonwoven material, apertured film or mixtures thereof.

8. The tampon of claim 7 wherein the nonwoven material comprises at least one of natural fibers, synthetic fibers and mixtures thereof.

9. The tampon of claim 8 wherein the natural fibers are at least one of rayon, cotton, wood pulp, flax, hemp or mixtures thereof.

10. The tampon of claim 8 wherein the synthetic fibers are at least one of polyester, polyolefin, nylon, polypropylene, polyethylene, polyacrylic, vinyl polyacetate, polyacrylate, cellulose acetate, or mixtures thereof.

11. The tampon of claim 10 wherein the synthetic fibers are bicomponent fibers that comprise at least two differing components of polyethylene, polypropylene or polyester.

12. The tampon of claim 1 wherein a withdrawal means is attached to the compressed absorbent member and extends beyond the withdrawal end.

13. A tampon having an outer surface comprising:
compressed absorbent member;
wherein the tampon comprises a finish that includes at least one rewetter chemical agent such that the tampon outer surface has a surface energy greater than about 42 mN/m.

14. The tampon of claim 13 wherein the tampon is a fibrous material.

15. The tampon of claim 14 wherein the finish is disposed on the fibers of the fibrous material.

16. The tampon of claim 13 wherein the finish comprises at least one chemical agent that is at least one of antistats, softeners, lubricants, optical brighteners or mixtures thereof

17. The tampon of claim 13 wherein the rewetter chemical agent comprises a surfactant that is at least one of a nonionic surfactant, anioinic surfactant, cationic surfactant, amphoteric surfactant, ampholytic surfactant or mixtures thereof.

18. The tampon of claim 13 wherein the compressed absorbent member comprises at least one of natural fibers, synthetic fibers or mixtures thereof.

19. The tampon of claim 18 wherein the synthetic fibers are at least one of polyester, polyolefin, nylon, polypropylene, polyethylene, polyacrylic, vinyl polyacetate, polyacrylate, cellulose acetate, or mixtures thereof.

20. The tampon of claim 19 wherein the synthetic fibers are bicomponent fibers that comprise at least two differing components of polyethylene, polypropylene or polyester.
